(19) United States
(12) Patent Application Publication
(10) Pub. No.: US 2003/0180391 A1
(43) Pub. Date: Sep. 25, 2003

(54) Plant drug for preventing cancer

(76) Inventor: Xinxian Zhao, Shenzhen (CN)

Correspondence Address: Xinxian Zhao, 67-08 168th Street, Flushing, NY, 11365, US

(21) Appl. No.: 10/078,587

(22) Filed: Feb. 20, 2002

(51) Int. Cl. A61K 35/78; A61K 31/44; A61K 31/70

(52) U.S. Cl. 424/725; 514/456; 514/280

(57) ABSTRACT

This invention relates to new safe plant drug, which contains Berberine and Baicalin (BB), inhibits carcinogenic and mutagenic action, lowers tyrosine kinase and decreases synthesis of DNA, RNA and protein of cancer cells.

Also, the present invention proved a new radioimmunoassay (RIA) method for precise determination of Berberine and Baicalin. The RIA is an efficient analytical method for large clinical programs including double blind analysis (DBA), and good clinical practice (GCP).

### BACKGROUND OF THE INVENTION

This invention relates to new safe plant drug, which used for prevention and treatment of cancer, specifically the drug inhibits carcinogenic and mutagenic action and lowers activity of tyrosine kinase, decreases synthesis of DNA, RNA and protein of cancer cells.

The new safe plant drug contains Berberine and Baicalin.

### DESCRIPTION OF PRIOR ART

Cancer is the second leading cause of death in the United States, and the incidence of cancer continues to climb annually. In recent years, about 1 million new cases of cancer are diagnosed yearly in the U.S. About half million people and 7 million people of annual deaths in the US and in the world, respectively

Many reports indicated that the side effects of plant's anticancer drugs are lower than chemical and antibiotic's anticancer drugs. Therefore, the development of plant drug has progressed very fast now. Taxol, for example, is a novel anticancer plant drug isolated from the needles and bark of the western yew, Taxus brevifolia. It is the prototype for a new class of antitumor drugs, which are characterized by their capacity to promote the assembly of microtubules. Clinical trials conducted in the late 1980s and early 1990s demonstrated impressive clinical activities against advanced ovarian and breast cancer.

However, taxol has two big problems. The first problem is that natural source of taxol is very limited. And the second problem is that taxol is a poor-water soluble. Vehicles for parental administration on taxol cause serious side effects.

The most remarkable progress in the last 25 years has been in cancer biology. Now we understand what is required to turn a normal cell into a cancer cell. Cancer arises when a single cell changes so that it divides continuously, released from the controls that constrain the replication of normal cells. This transformation is due to changes in the function and activity of genes, which are segments of DNA. Of the 100,000 genes found in the human genome, only the altered activities of a small number of genes are responsible for transforming a normal cell into a cancer cell. These genes normally function to instruct cells to produce accelerators that drive cells to proliferate, brakes that control proliferation, or the repair of DNA damage or the elimination of damaged cells.

We now know that DNA changes are the fundamental cause of all cancers. These changes can occur due to chemicals, viruses, radiation, and mistakes of duplicating DNA. When a normal cell recognizes damage to its DNA, it stops the process of growth and division. In the development of cancer, checkpoint controls are lost and the cell continues to divide, transmitting its damaged DNA to new cells.

Cancer does not develop at short time. The molecular changes necessary to transform a normal cell into a cancer cell may take years to accumulate. This is one reason for prevention of cancer by administering drugs that inhibit the crucial molecular events causing transformation. The present invention disclosed that control process of turning a normal cell into a cancer cell by special natural and safe drugs. Recently a number of gene expression systems have been developed that can be regulated by the administration of specific small molecule drugs. It is important that the small molecule drugs are easily administered. Some of the earliest systems include exposure to heavy metal, and steroid hormones, but they are not suited for in vivo human. These systems, however, are suitable to study for the effect of small molecular drugs on controllable oncogenes, and to study for the treatment of cancer.

So far, no one drug has been succeeded to treat or prevent cancer by control cancer cells and without adverse side effects.

### DETAILED DESCRIPTION OF THE INVENTION

The inhibiting carcinogenic and mutagenic action, lowering activity of tyrosine kinase, decreasing synthesis of DNA, RNA and protein of cancer cells is focus in cancer biology and molecular pharmacology of anticancer drug.

The compositions of drugs and the sources of its components are listed as below.

TABLE 1

| Component | Source |
|---|---|
| Berberine (BE) | Berberis poiretii Schined |
| | Berberis julianae Schneid |
| | Berberis soulieana Schined |
| | Berberis wilsonae Schined |
| Baicalin (BA) | Scutellaria baicalensis Georgi |
| | Scutellaria scordifolia Fisch |

TABLE 2

| | | Preferred composition |
|---|---|---|
| Component | Weight percent | weight percent |
| BE | 30-70 | 50 |
| BA | 30-70 | 50 |

The drug, which is mixed BE and BA, named “BB”.

Berberine (BE) and Baicalin (BA) have the following chemical structures:

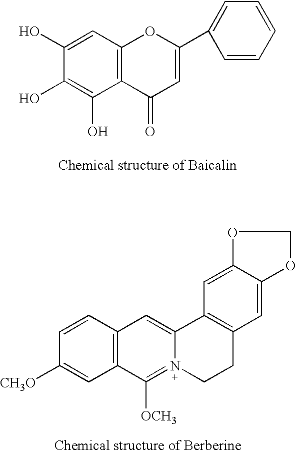

The following specific examples will provide detailed illustrations of methods of producing relative drugs, according to the present invention and pharmaceutical dosage units containing relative drugs. Moreover, examples described pharmaceutical characters of drugs, which demonstrated its effectiveness in control of cancer cells. These examples are not intended, however, to limit or restrict the scope of the invention in any way, and should not be construed as providing conditions, parameters, reagents, or starting materials which must be utilized exclusively in order to practice the present invention.

### EXAMPLE 1

### Mutagenic Effect of BB

Determination of the mutagenic and carcinogenic activity is important for estimating side effects of drug. The mutagenic activity of many drugs can only be detected with growing cells. In present study, mutagenic and carcinogenic activity of BB is determined by Bacteria system. The method for detecting mutagenicity of BB, with the Salmonella system that detects the reversion of the bacteria from His⁻ to His⁺, is widely used.

Methods for detecting carcinogens and mutagens with the salmonellia mutagenicity test are highly efficient in detecting carcinogens and mutagens. Major carcinogens tested have been detected as mutagens. Salmonella mutagenicity assay is very sensitive and simply test for detecting mutagens and carcinogens. Therefore, it has been useful in a detailed study that has been made of mutagenic activity of BB.

TAa7, TAa8, TA100 and TA102 are extremely effective in detecting classes of carcinogens and mutagenesis.

The bacterial tester strains used for mutagenesis testing are TA97, TA98, TA100 and TA102. Mutagenesis testing method was done as described previously. In brief, TA97, TA98, TA100 and TA102 were grown in agar gel culture. The petri plats (100×15 mm style) contain 30 ml with 2% glucose. The agar mixture was agitated vigorously and immediately poured into plates of minimal agar. The cultures were incubated at 37° C. in a dark and 5% CO₂ in air for 48 hours. After 48 hours the colonies in both test and controls are counted. The presence of a background lawn of bacteria on the histidine-poor soft agar plate was used as an indication that gross toxic effects were absent. Mutagenicity assays were carried out at least in triplicate.

The data of experiment summarized as the following table.

TABLE 3
Mutagenesis Assay on plates

| | Dose/ | Number of His⁺ revertants/plate | | | | | |
|---|---|---|---|---|---|---|---|
| | plate | TA97 | | TA98 | | TA100 | |
| Treatment | (μg) | −S | +S | −S | +S | −S | +S |
| Spontaneous | — | 149 ± 15 | 150 ± 17 | 35 ± 4 | 36 ± 4 | 120 ± 17 | 120 ± 15 |
| 4NQO | 0.5 | 861 ± 79 | — | 338 ± 35 | — | 2301 ± 190 | — |
| BB + 4NQO | 100 | 280 ± 16 | — | 138 ± 4 | 82 ± 4 | 298 ± 15 | 280 ± |
| BB | 10 | 160 ± 16 | 185 ± 16 | 55 ± 4 | 56 ± 3 | 152 ± 17 | 155 ± |

*4QO: 4-nitroquinoline-1-Oxide

The salmonella typhimurium strains TA97, TA98 and TA100 were checked using 4-nitroquinoline-1-oxide. The range of spontaneous mutation rates for the individual strains, which were considered to be acceptable, was TA97 (100-170), TA98 (20-40) and TA100 (80-150).

The data of Table 20 indicated that the number of His+ revertants/plate of BB almost is as same as spontaneous of testing strains. On the contrary, 4NQO is mutagenic and carcinogenic agent.

The number of His+ revertants/plate of 4NQO is higher than 10 times of spontaneous.

In conclusion, BB strongly inhibits carcinogenic and mutagenic action.

### EXAMPLE 2

### Inhibition of Tyrosine Kinase Activity by BB

In general, very low levels of tyrosine kinase (TK) are expressed in normal cells and high levels of TK are expressed in cancer cells. Therefore, a drug, which inhibits the activity of TK, can provide a new way to overcome cancer. In other words, the development of effective inhibitors of TK can be used for the treatment of cancer.

Materials and Methods

[³²P]ATP and other isotopes were purchased form Amersham Corp. All other chemicals were reagent grade obtained from commercial suppliers.

Tyrosine kinase (TK) Assay: TK was measured by a modification of the method of Braun et al. ⁽⁴⁴⁾. Briefly, H-60 leukemia cells were plated at a density of 5×1 cells in 60-nm dished, and divided control and treatments groups for incubation 24 hours at 37° C. with 5% CO₂. The cells were collected by scraping, washed twice with phosphate-buffered saline, and resuspended at density of 10⁶ cells/ml in 5 mM HEPEs buffer (pH 7.4). The cells were then resuspended in 1 ml of buffer containing 5 mM HEPES (pH 7.6), 1 mM MgCl₂ and 1 mM EDTA, then placed on ice bath. The cell membrane was disrupted by ultra sound and centrifuged at 1000×g for 10 minutes. The supernatant was ultra centrifuged at 30,000×g for 30 minutes at 4° C. The pellet was resuspended in 0.3 ml of buffer containing 25 mM HEOES, centrifuged at 12,000×g for 5 minutes. The resulting supernatant was used for TK assay. Content of protein was determined. 10 μg of protein placed in 20 mM HEPES (pH 7.6), 15 mM MgCl₂, 10 mM ZnCl₂ and P-40, with or without substrate [glutamic acid (GT), mg/ml]. After 5 minutes incubation at 25° C., the reaction was initiated by the addition of 25 μM [γ³²P] ATP (3 ci/mmol). After 10 minutes, the reaction was stopped by the addition of 20 mM cold ATP. 50 μl of the mixtures were spotted on glass microfibers filter discs and washed three times with cold trichloroacetic acid (TCA), contained 10 mM sodium pyrophosphate. Air dried. Radioactivity was determined by liquid scintillation spectrometry. The net TK activity was determined after correcting for endogenous TK activity.

The present study clearly demonstrated that BB reduction in TK activity. A concentration-dependent inhibition was seen. BB caused a relatively strong inhibition.

TABLE 4
Effect of BB on TK activity of HL-60 leukemia cells

| Drugs | Concentration (M) | % of control activity |
|---|---|---|
| None | — | 100 |
| BB (1) | 10⁻⁶ | 4.2 |
| BB (2) | 10⁻⁷ | 28.5 |

Value represents the mean of three experiments each done in duplicate; the range was less than % of the mean.

BB inhibited the HL-60 TK activity by 4.2% (10⁻⁶ M) and 28.5% (10⁻⁷ M) of control activity. Therefore, BB can significantly inhibit TK activity.

Evidences have accrued demonstration that TK is important in regulation of cellular proliferation, differentiation, maturation, canceration and carcinogens. TK inhibitors should be used in investigation of the mechanism of carcinogenesis and they may be effective in prevention and chemotherapy of cancer. The results of experiments described on this paper are paralleled our previous works. BB is a screened using chemoprevention-associated biochemical methods by National Cancer Institute of the US (NIH).

In conclusion, BB strongly inhibits TK of cancer cells.

### EXAMPLE 3

### Inhibition of Protein Biosynthesis by BB

BB powder with purity greater than 98% was extracted from Cephalotaxus harringtonia at Shanghai Pharmaceutical Institute. BB stock solution (2×10⁻³M) was dissolved in 0.001 N HCl, filtration sterilized, and stored frozen at −15° C. Working concentration of BB was made by dilution of the stock solution with RPMI-1640 medium. [¹⁴C] leucine (200 Ci/mmol) was purchased from New England Nuclear Corporation. All other chemicals were reagent grade obtained from commercial suppliers.

L1210 and P388 cells were grown at 37° C. on medium RPMI-1640 without antibiotics and supplemented with 10% horse serum. Cultures were diluted daily to 1×10⁵ cells/ml with fresh growth medium. From a culture initiated with cells from ascitic fluid obtained from a mouse 5 days after implantation with in vivo-passaged leukemia, a stock of ampoule containing 10⁷ cells/ml in growth medium plus 10% dimethyl sulfoxide was frozen and stored in liquid nitrogen. Cultures were started from the frozen stock and were passaged for no more than 1 month.

L1210 and P388 cells were grown at 37° C. on medium RPMI-1640 (sigma) supplemented with 10% calf serum, 10,000 unit/ml of Penicillin and 10,000 unit/ml of Streptomycin. 1×10⁶/ml cells were placed in culture with different concentrations of BB. Then cells mixed with 2 μM [¹⁴C]-leucine (200mCi/mmole) immediately, and the cell suspension was incubated at 37° C. in a humidified atmosphere of 5% CO₂-95% air for the indicated time. Reactions were terminated by addition of 3 ml of cold Earle's buffer. Cells were lysed, precipitated with 10% trichloroacetic acid (TCA) and filtered onto glass fiber filters. The filters were washed with phosphate-buffered saline and placed in scintillation vials, and radioactive emissions were counted.

Adult DBA/2J male mice, 6 to 8 weeks old. All animals weighed approximately 25 g when used in experiments. Mice were assigned randomly to treatment and control groups. Each member of which received identical dosed (i.p.) of BB or 0.9% NaCl solution for all injections. Volumes were 0.01 ml/g body weight.

Tumor: P388 leukemic cells induced in mice by inoculation with or P388 leukemic cells (1.0×10⁵). Ascitic fluid was collected aseptically and diluted medium. Only ascitic fluid, which was free of erythrocytes, was used since the presence of erythrocytes inhibited growth of the leukemic cells.

The leukemic cells were passed i.p. in weekly. P388 cells were cultured at 37 C in RPMI-1640 medium (GIBCO) without antibiotics and supplemented with 10% fetal calf serum.

TABLE 5
Effect of varying concentrations of BB on synthesis of protein of
L1210 in leukemia Cells.

| Group | | CPM | P* |
|---|---|---|---|
| Control | — | 24585 | — |
| BB (ng/ml) | 10 | 6240 | <0.01 |
| BB (ng/ml) | 20 | 4300 | <0.001 |

*P is different between control group and treatment group.

TABLE 6
Effect of varying times of BB on synthesis of protein of
L1210 in leukemia cells

| | CPM | | |
|---|---|---|---|
| Time (min.) | C | T | P |
| 5 | 1290 | 387 | <0.01 |
| 60 | 9200 | 3200 | <0.01 |
| 120 | 12045 | 4210 | <0.001 |

The data of the present experiment show that BB is a very effective inhibitor of protein synthesis of cancer cells.

Tumor and BB used in this study have been described on section of “Therapy of mouse leukemia L1210 and P388 by homoharringtonine”. L1210 leukemia cells were cultured in Hank's balanced salt solution (HBSS) from the ascitic aspirates of DBA/2J mice 7 days post-inoculation with 1×10⁵ cells i.p. The cells were washed 3 times to remove the heparin and ascitic fluids and resuspended in HBSS containing 33% fetal calf serum (FCS) to a concentration of 1×10⁵ cells/ml. Leukemia cells were grown at 37° C. in medium 1640 containing 10% of FCS, without antibiotics.

The growing cells were suspended in fresh grown medium at a density of 1×10⁵ cells/ml. The cells suspensions were warmed to 37° C. for 20 minutes and divided into 1 ml portions. For measurement of BB effects the cells suspensions were mixed with 20 μl BB. BB concentration of treatment group is 0.5 μg/ml and cells of control group mixed with same volume of 0.9% Nacl solution. Then cells of two groups mixed with 0.5 μCi [³H]-TdR (20 μCi/mM) or 0.5 μCi [³H]-U (20 μCi/mM) immediately. Incubation times were 20 minutes at 37° C. after the addition of [³H]-TdR or [³H]-U.

At the end of the incubation time the cells were washed twice with cold 0.9% NaCl solution. 3 ml of 10% trichloroacetic acid were added and the resultant precipitate was washed two with trichloroacetic acid. The acid-precipitable material was dissolved in 1.0 ml NCS reagent and counted in 15 ml of scintillation mixture (toluene containing 30% ethanol, 0.6% PPO and 0.03% POPOP) in liquid scintillation system to counting error of 1% or less.

TABLE 7
Inhibition of BB on ³H-TdR into DNA

| | ³H-TdR uptake | Inhibition of BB on | |
|---|---|---|---|
| Group | (CPM) | ³H-TdR into DNA | P |
| Control | 5800 ± 650 | — | — |
| (0.9% NaCl) | | | |
| Treatment | 2912 ± 320 | 50.0% | <0.001 |
| (BB 0.5 μg/ml) | | | |

TABLE 8
Inhibition of BB on ³H-U into RNA

| | ³H-TdR uptake | Inhibition of BB on | |
|---|---|---|---|
| Group | (CPM) | ³H-U into RNA | P |
| Control | 50590 ± 7011 | — | — |
| (0.9% NaCl) | | | |
| Treatment | 42961 ± 5200 | 15.0% | <0.01 |
| (BB 0.5 μg/ml) | | | |

This study suggested that effect of BB in leukemic cell effectively inhibited DNA synthesis (see Table 7); [³H]-TdR incorporation into DNA and [³H]-U incorporation into RNA in L1210 cells were inhibited 50% and 15% within 20 minutes by 0.5 μg/ml of BB respectively.

In conclusion, BB can inhibit DNA and RNA synthesis of cancer cells.

### EXAMPLE 5

### Extraction of Berberine

Berberine was extracted from Berberis poivetii Schined or Berberrros julianae Schined. The roots of plant dried and powdered. 3 liters of 0.1% H₂SO₄ was added to 1 kg of dried powder and allowed to stand for one day at room temperature. The solution was filtered and extracted filtrate saved. 2000 ml of 0.1% H₂SO₄ added to the residue, and extracted was repeated. The filtrate combined. HCl added to filtrate and adjusted to pH 1.5. NaCl added the solution of HCl and adjusted concentration of NaCl to 10% with stir. And allowed to stand for two days at room temperature. The solution was filtered and the residue was saved. The hot water (60° C.) added to the residue and suspension obtained. Ca (OH)₂ was added to suspension and adjusted pH to 8.5. Solution of Ca (OH)₂ was filtered and filtrate saved. HCl added to filtrate and adjusted pH to 1.5 and allowed for standing 2 hours at room temperature. Precipitate was obtained. Precipitate was filtered and the residue saved. Residue was washed by distilled water. Dried under vacuum. Powdered. The final product is Berberine.

### EXAMPLE 6

### Extraction of Baicalin

Baicalin was extracted from the roots of Scutellaria baicalensis George. The roots of plant dried and powdered. 10 liters water (80° C.) was added to 1 kg of dried powder and allowed standing a half of hour. The extraction was repeated twice by collecting the hot water, replacing it with an equal volume of water (80° C.). The water was combined. HCl added to water and adjusted pH to 1.5 and allowed standing for a half of hour at 80° C. The solution filtered and residue saved. The residue washed by 95% ethanol. The residue filtered under vacuum. Water added to residue and adjusted pH to 6.8 by 40% NaOH. Active carbon added to water solution of NaOH and allowed standing for a half of hour at 80° C., then allowed standing for 10 hours at room temperature. The precipitant filtered under vacuum. The precipitant was washed by 95% ethanol and dried at 60° C.

### EXAMPLE 7

### BB Injecting Preparation

50% of BE and 50% of BA, according to the conventional methods, was made as ampoules or other injection preparation, then sterilized. Type XGI.S double door functional ampoule sterilizing machine is used for manufacturing of BB injection. The function of facilities includes sterilization, leakage detection and washing. Microcomputer (PC machine) is applied in the principal controlling system. Dose is intramuscularly 5-100 mg daily.

### EXAMPLE 8

### BB Oral Preparation

50% of BE and 50% of BA powder granulated accorded to the conventional granulation method. The mixture content decreased from 100% to 93%. The 7% of content was different types of fillers. Disintegrants and lubricants were used: microcrystalline cellulose (Avicel PH 105, PH 101, PH 102, PH 200, all from FMC Corp., Lehmann and Voss and Co., Hamburg, Germany; and Vivacel 200, Rettenmaier and Söhne GmbH, Ellwangen-Holzmühle, Germany), microfine cellulose (Elcema P 050, P 100, G 250, all from Degussa AG, Frandfurt, Germany; and Tablettierhilfsmittel K, Merck KGaA, Darmstadt, Germany), lactose cellulose granulate (Cellactose, Meggle, Wasserburg, Germany), α-lactose monohydrate (Lactose D 80, Meggle, Wasserburg, Germany), and modified maize starch (Starch 1500, Colorcon GmbH, Königstein, Germany).

The disintegrants tested were the following: cross-linked sodium carboxymethyl cellulose (Ac-Di-Sol, FMC Corp./Lehmann and Voss and Co.; and Nymcel ZSB 10, Nymcel ZSB 16, METSÄ-SERLA, Njimegen, Netherlands), Cross-linked calcium carboxymethyl-cellulose (ECG 505, FMC Corp./Lehmann and Voss and Co.), potato starch (Caeleo, Hilden, Germany), sodium starch glycolate (Explotab, Gustav Parmentier, Frankfurt, Germany; and Primojel, AVEBE Deutschland, Düsseldorf, Germany), cross-linked polyvinylpyrrolidone (Kollidon CL, BASF AG, Ludwigsburg, Germany; and Polyplasdone XL, ISP Deutschland, Frechen, Germany), and low-substituted hydroxypropyl-cellulose (L-HPC LH 22, L-HPC LH 31, both from Shin-Etsu Chemical Co., Ltd., Tokyo, Japan).

For lubrication, the following were used: magnesium stearate (Otto Bärlocher GmbH, Munich, Germany), glyceryl tristearate (Dynasan 118, Hüls Ag, Witten, Germany), and polyethylene glycol (PEG 6000, Hoechst AG Frankfurt/Main, Germany).

Colloidal silicon dioxide (Cab-O-Sil M 5, Cabot GmbH, Hanau, Germany; Syloid 244, W. R. Grace and Co., Lexington, Ky., and Aerosil 200, Degussa AG, Frankfurt/Main, Germany) and hydrophobic colloidal silicon dioxide (Aerosil R 972, Degussa AG) were used. As a stabilizer, ascorbic acid (Merck KGaA, Darmstadt, Germany) was added.

The content of BB was kept constant at a level of 100 mg per tablet. Tablet weight was varied between 100-105 mg. Tablet mixtures were mixed for 10 min in the Turbula mixer (type T2C, Willy Bachofen, Basel, Switzerland). The n lubricants were sieved through a 315-μm sieve into the mix. Final mixing was carried out for 5 min at 42 rpm in the Turbula mixer. The mixtures were compressed using a rotary press (Korsch PH 103, Korsch, Berlin). The lower compression roller was instrumented with four strain gauges (type 3/120 LY 11, Holtinger Baldwin, Inc., Darmstadt, Germany). A Philips carrier-frequency bridge (PR 9307 Philips, Kassel, Germany) was used for signal amplification. Each batch was compressed at different levels of compression force in the range of 1 to 25 kN. As a stabilizer, ascorbic acid (Merk KGaA, Darmstadt, Germany) was added. Sugar-coating operation was also performed conventionally. The dosage of BB is orally 50-200 mg daily.

### EXAMPLE 9

### Radioimmunoassay for the Quantitative Determination of Berberine

A radioimmunoassay for the determination of the pilogram (10⁻¹² g) amounts of Berberine has been developed. The measuring range of the assay extends from 0.05 to 20 ng of Berberine. This assay allows the rapid, sensitive and precise determination of Berberine. Berberine is currently being determined by a number of methods including thin-layer chromatography. The sensitivity of thin-layer methods is in the microgram range. Also only a few of samples can be analyzed per day. In the past years, the radioimmunoassay (RIA) has proven to be very useful for the determination of various plant constituents. This method, using the sensitivity and selectivity of high-affinity antibodies together with tracer antigens of very high specific activity, allows the precise measurement of very low concentrations of compounds in manufacture process, crude plant extracts and in blood of patients who treated with Berberine. More important, the RIA method is allowed the analysis of up to more than five hundreds per day. Therefore, RIA is an efficient analytical method for large clinical programs, including double blind analysis (DBA) and good clinical practice (GLP). GLP and DBA are requested by FDA for approval a new drug.

The present invention disclosed that in a high specific RIA of Berberine, which allows the precise quantification of Berberine in the pilogram (10⁻¹² g) range.

Method: ³H-Berberine was synthesized. Berberine (1 g) and hemisuccinate (1 g) were dissolved in 2.5 ml pyridine and the solution was refluxed for 1 hour. The solution was diluted with 7.5 ml H₂O and the pH was adjusted to 4 by 1N HCl. The solution was extracted 10 ml CHCl₃ with three times. The CHCl₃-phases were pooled and concentrated to 1 ml under vacuum. The concentrate mixed with 2 ml petrol ether (40°-60° C.). The precipitate was separated by filtration and recrystallized from MeOH yielding Berberine hemisuccinate.

50 mg Berberine hemisuccinate dissolved in 5 ml of 50% of aqueous C₅H₅N. Added 50 mg of 1-ethyl-3-(3-dimethylaminopropyl)-carbodiimide hydrochloride in 50% aqueous C₅H₅N with stirring at room temperature. 82 mg of solution of bovine serum albumin (BSA) was added to aqueous C₅H₅N and then mixture was stirred for 24 hours at room temperature. The mixture dialysed against H₂O for 4 days, and Berberine hemisuccinate conjugate.

The conjugate was administrated to rabbits. After 4 weekly intradermal immunizations, intramuscular booster injections were given monthly. The blood was collected 1 and 2 weeks after each booster. The whole cells were removed by centrifugation. The antiserum was collected and stored at −20° C. Samples and standards were added to glass tubes. ³H-Berberine (5000 cpm in 0.1 ml) and H₂O were added to tubes. Incubation was started by addition of antiserum. The samples and standards were incubated for 1 hour at room temperature following by the addition of 1 ml of 98% solution of freshly prepared (NH₄)₂SO₄ and mixing. The samples and standards were further incubated for 1 hour at 20° C. and then centrifuged (10 min at 4000 rpm). The pelles were washed with 50% of (NH₄)₂SO₄ then dissolved in 0.25 ml of H₂O and mixed with 1 ml of scintillation. The tubes were counted for radioactivity. The results RIA-calculations were done on a corrected count by linear interpolation from the standard curve, after correction for dilution.

### EXAMPLE 10

### Radioimmunoassay for the Quantitative Determination of Baicalin

Method: ³H-Baicalin was synthesized. Baicalin (1 g) and hemisuccinate (1 g) were dissolved in 2.5 ml pyridine and the solution was refluxed for 1 hour. The solution was diluted with 7.5 ml H₂O and the pH was adjusted to 4 by 1N HCl. The solution was extracted 10 ml CHCl₃ with three times. The CHCl₃-phases were pooled and concentrated to 1 ml under vacuum. The concentrate mixed with 2 ml petrol ether (40°-60° C.). The precipitate was separated by filtration and recrystallized from MeOH yielding Baicalin hemisuccinate.

50 mg Baicalin hemisuccinate dissolved in 5 ml of 50% of aqueous C₅H₅N. Added 50 mg of 1-ethyl-3-(3-dimethylaminopropyl)-carbodiimide hydrochloride in 50% aqueous C₅H₅N with stirring at room temperature. 82 mg of solution of bovine serum albumin (BSA) was added to aqueous C₅H₅N and then mixture was stirred for 24 hours at room temperature. The mixture dialysed against H₂O for 4 days, and Baicalin hemisuccinate conjugate. The conjugate was administrated to rabbits. After 4 weekly intradermal immunizations, intramuscular booster injections were given monthly. The blood was collected 1 and 2 weeks after each booster. The blood were removed whole cells by centrifugation. The antiserum was collected and stored at −20° C. Samples and standards were added to glass tubes. ³H-Baicalin (5000 cpm in 0.1 ml) and H₂O were added. Incubation was started by addition of antiserum. The samples and standards were incubated for 1 hour at room temperature following by the addition of 1 ml of 98% solution of freshly prepared (NH₄)₂SO₄ and mixing. The samples and standards were further incubated for 1 hour at 20° C. and then centrifuged (10 min at 4000 rpm). The pelles were washed with 50% of (NH₄)₂SO₄ then dissolved in 0.25 ml of H₂O and mixed with 1 ml of scintillation. The tubes were counted for radioactivity. The results RIA-calculations were done on a corrected count by linear interpolation from the standard curve, after correction for dilution.

The preparation of drugs which can be accomplished by the extraction methods set forth above or any conventional methods for extracting the active principles from the plants. The novelty of the present invention resides in the mixture of the active principles in the specified proportions to produce drugs, and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, capsules, tablets, syrups, elixirs, and solutions for parenteral injection with specified ranges of drugs concentration.

In addition, the present invention provides novel methods for treating and preventing a variety of cancer conditions and control cancer cells with produced safe pharmaceutical agent.

It will thus be shown that there are provided compositions and methods which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letter Patent is set forth in the appended claims:

1. A safe plant drug for prevention and treatment of cancer comprises Berberine and Baicalin.

2. A safe botanical drug of claim 1 wherein the amount to sufficient to prevent and treat cancer is orally about 25-300 mg of BB which contained 50% of Berberine and 50% of Baicalin.

3. A safe botanical drug of claim 1 wherein the amount to sufficient to inhibit mutagens is about 25-300 mg of BB which contained 50% of Berberine and 50% of Baicalin.

4. A safe botanical drug of claim 1 wherein the amount to sufficient to inhibit carcinogens is about 25-300 mg of BB which contained 50% of Berberine and 50% of Baicalin.

5. A safe botanical drug of claim 1 wherein the amount to sufficient to decrease activity of tyrosine kinase is about 25-300 mg of BB which contained 50% of Berberine and 50% of Baicalin.

6. A safe botanical drug of claim 1 wherein the amount to sufficient to decrease protein synthesis of cancer cells is about 25-300 mg of BB which contained 50% of Berberine and 50% of Baicalin.

7. A safe botanical drug of claim 1 wherein the amount to sufficient to decrease RNA synthesis of cancer cells is about 25-300 mg of BB which contained 50% of Berberine and 50% of Baicalin.

8. A safe botanical drug of claim 1 wherein the amount to sufficient to decrease DNA synthesis of cancer cells is about 25-300 mg of BB which contained 50% of Berberine and 50% of Baicalin.

9. A method of RIA used for determination of Berberine is comprising:
a. ³H-Berberine was synthesized;
b. Berberine and hemisuccinate were dissolved in pyridine and solution was refluxed for 1 h;
c. the solution was diluted with H₂O;
d. the solution was extracted with CHCl₃;
e. the CHCl₃-phases were pooled and concentrated under vacuum;
f. the concentrate mixed with petrol ether;
g. the precipitate was separated by filtration and recrystallized from MeOH, yielding Berberine hemisuccinate;
h. the Berberine hemisuccinate dissolved in 50% of aqueous C₅H₅N;
i. added 1-ethyl-3-(3-dimethylaminopropyl)-carbodiimide hydrochloride in 50% aqueous C₅H₅N with stirring;
j. solution of bovine serum albumin was added to aqueous C₅H₅N and the mixture was stirred;
k. the mixture dialysed against H₂O and Berberine hemisuccinate conjugate;
l. the conjugate was administrated to rabbits then intradermal immunizations, intramuscalar booster injections were given;
m. the blood was collected after each booster and whole cells were removed by centrifugation;
n. the antiserum was collected and stored;
o. samples and standards were added to tubes and then BSA, ³H-Berberine and H₂O were added to tubes;
p. the incubation was started by addition of antiserum, the samples and standards were incubated following by the addition of fleshly prepared (NH₄)₂SO₄ solution and mixing;
q. the samples and standards were further incubated and centrifuged;
r. the pelles were washed with (NH₄)₂SO₄ and dissolved in H₂O then mixed with scintillation;
s. The tubes were counted for radioactivity; and
t. the RIA-calculations were done on a corrected count by linear interpolation from the standard curve after correction for dilution.

10. A method of RIA used for determination of Baicalin is comprising:
a. ³H-Baicalin was synthesized;
b. Baicalin was dissolved in pyridine and solution was refluxed for 1 h;
c. the solution was diluted with H₂O;
d. the solution was extracted with CHCl₃;
e. the CHCl₃-phases were pooled and concentrated under vacuum;
f. the concentrate mixed with petrol ether;
g. the precipitate was separated by filtration and recrystallized from MeOH, yielding Baicalin;
h. the Baicalin dissolved in 50% of aqueous C₅H₅N;
i. added 1-ethyl-3-(3-dimethylaminopropyl)-carbodiimide hydrochloride in 50% aqueous C₅H₅N with stirring;
j. solution of bovine serum albumin was added to aqueous C₅H₅N and the mixture was stirred;
k. the mixture dialysed against H₂O and Berberine hemisuccinate conjugate;
l. the conjugate was administrated to rabbits then intradermal immunizations, intramuscalar
m. booster injections were given;
n. the blood was collected after each booster and whole cells were removed by centrifugation;
o. the antiserum was collected and stored;
p. samples and standards were added to tubes and then BSA, ³H-Baicalin and H₂O were added to tubes;
q. the incubation was started by addition of antiserum, the samples and standards were incubated following by the addition of fleshly prepared (NH₄)₂SO₄ solution and mixing;
r. the samples and standards were further incubated and centrifuged;
s. the pelles were washed with (NH₄)₂SO₄ and dissolved in H₂O then mixed with scintillation;
t. the tubes were counted for radioactivity; and
u. the RIA-calculations were done on a corrected count by linear interpolation from the standard curve after correction for dilution.
